(19) United States
(12) Patent Application Publication
(10) Pub. No.: US 2008/0070990 A1
(43) Pub. Date: Mar. 20, 2008

(54) USE OF RACTOPAMINE ENANTIOMERS

(75) Inventors: Calvin London, Perth (AU); A.K. Gunner Aberg, Sarasota, FL (US)

Correspondence Address: HOWREY LLP, C/O IP DOCKETING DEPARTMENT, 2941 FAIRVIEW PARK DR, SUITE 200, FALLS CHURCH, VA, 22042-2924, US

(73) Assignee: STIRLING PRODUCTS LIMITED, Perth (AU)

(21) Appl. No.: 11/747,761

(22) Filed: May 11, 2007

Related U.S. Application Data
(60) Provisional application No. 60/809,205, filed on May 30, 2006.

(51) Int. Cl. A61K 31/135; A23K 1/16; A61P 21/00; A61P 3/04; A23K 1/17

(52) U.S. Cl. 514/653; 426/532; 426/648

(57) ABSTRACT

A method of promoting or improving the feed efficiency and the muscle to fat ratio livestock animal by administering to the animal a therapeutically effective amount of a pure or substantially pure RR-enantiomer of ractopamine. Also disclosed are animal feed preparations and compositions and pharmaceutical preparations capable of increasing lean meat deposition in an animal or for improving lean meat to fat ratio in an animal or for promoting or improving the growth of an animal or for improving the feed efficiency of an animal, the feed preparation, compositions and pharmaceutical preparations including a therapeutically effective amount of a pure or substantially pure RR-enantiomer of ractopamine.

The present application claims the benefit of priority of U.S. Provisional Application No. 60/809,205, which was filed May 30, 2006. The entire text of the aforementioned application is incorporated herein by reference in its entirety.

### FIELD OF THE INVENTION

The invention relates to methods of increasing the muscle/fat ratio in an animal, promoting or improving the growth of an animal and/or improving the feed efficiency of animals by administering therapeutically active enantiomers of ractopamine or a derivative thereof to the animals in a quantity which is effective for this purpose. The invention further relates to compositions for use in the methods and to animal feed additives, which comprise one or more therapeutically active enantiomers of ractopamine as the active substance.

### BACKGROUND OF THE INVENTION

Adrenergic beta-agonistic drugs characteristically contain as part of their structure an ethanolamine or 2-amino-ethanol moiety. Since the chemical structures of these drugs usually comprise at least one asymmetric carbon atom, these drugs commonly exist in optically active isomeric form, with the chiral carbon atom having the (R) or (S) configuration [as designated using the Cahn-Ingold-Prelog system (Angew. (1966) Chem. Item. Ed. 5, 885-415). When there is a sole asymmetric carbon atom present, the beta-receptor agonist exist as individual (R) or (S) enantiomers or in racemic [(RS)] form, i.e. as a 50:50 mixture of the (R) and (S) enantiomers.

Compounds with two chiral centers have four enantiomers: the RR-, SS-, RS-, and SR-enantiomers. Such compounds (e.g. ractopamine) may exist in a number of forms i.e. in individual RR, SS, RS or SR isomeric forms, as racemic mixtures comprising, for example, RR plus SS or RS plus SR enantiomeric pairs, as well as in the form of diastereomeric mixtures comprising all four isomeric forms.

Many biologically active molecules exist as enantiomers. Although structurally identical, enantiomers can have different effects in biological systems: one isomer may have specific therapeutic activity while the other isomer may have no therapeutic activity or may have entirely different forms of biological activity.

An example of a compound with two chiral centers is ractopamine. The racemic form of ractopamine is commercially available under the trade names Paylean®, Elanco and Optaftex®, Elanco and is used as a growth promotant (i.e., growth promoter) for livestock.

Ractopamine has the molecular formula C₁₈H₂₃NO₃ and is typically prepared as a hydrochloride salt. Ractopamine HCI (4-hydroxy-a-[[[3-(4-hydroxyphenyl)-1-methylpropyl]amino]methyl]benzenemethanol hydrochloride) has a molecular weight of 337.85 and a molecular formula of C₁₈H₂₃NO₃.HCI (CAS number: 90274-24-1). The racemate is a mixture of its four enantiomers in approximately equal proportions.

Most adrenergic beta-receptor agonists have affinity for various types of beta receptors. Thus, as shown below, both R-salbutamol and ractopamine have affinity for beta-1 and beta-2 receptors, but negligible affinity for beta-3 receptors. While ractopamine has selective affinity for beta-1 receptors, R-salbutamol has selectivity for beta-2 receptors.

| | Affinity (Ki) for adrenergic β-receptors | | |
|---|---|---|---|
| Compound | β₁(h) | B₂ (h) | B₃ (h) |
| Ractopamine | 2.6E−07 | 3.0E−07 | 1.4E−05 |
| R-salbutamol | 3.3E−06 | 3.7E−07 | 5.2E−04 |

R-salbutamol was obtained from Dr. Y. Hamied, Cipla, Mumbai, India. Ractopamine was extracted from commercial Paylean® Elanco.

The pharmacological activity of a beta-receptor agonist like ractopamine is to activate adrenergic beta-receptors. Activation of adrenergic beta-receptors in animals including humans, warm-blooded animals and in fish leads to increased intracellular concentration of cyclic adenosine monophosphate (cAMP), which triggers various events in various cells and organs. Cellular responses to beta-receptor activation include for example lipolytic activity in adipose tissues, smooth muscle relaxant activity in the bronchi and increased frequency of contractions in the heart (Goodman-Gilman, The Pharmacological Basis of Therapeutics 9^{th} Ed., 1990, McGraw-Hill ISBN0-07-026266-7).

Of the four stereoisomers of ractopamine, which are RR-, RS-, SR- and SS-ractopamine, it is known that RR-ractopamine is the most potent, both when tested in vitro (Mills S E, Kissel J, Bidwell C A, Smith D J Stereoselectivity of porcine β-adrenergic receptors for ractopamine stereoisomers. J. Anim. Sci. 2003, 81: 122-129) and in vivo (Ricke E A, Smith D J, Fell V J, Larsen G L, Caton J S Effects of ractopamine HCl stereoisomers on growth, nitrogen retention and carcass composition in rats. J. Anim. Sci. 1999, 77:701-707.) Thus, when tested for binding affinity for porcine adrenergic β-2 receptors, RR-ractopamine was twice as active as RR/RS/SR/SS ractopamine (Mills at al. J. Anim. Sci. 2003. 81:

Importantly, beta-receptor agonist drugs have pharmacological and toxicological side effects that range from minor importance all the way through to major importance. For example, racemic ractopamine has been found to cause stress in livestock animals (Marchant-Forde J. N., et. al. 2003: “The effects of ractopamine on the behaviour and physiology of finishing pigs” J Anim Sci., 81: 416-422). This is a side effect of major importance, as it is considered to be a reason for increased heart rate and stress levels in animals during handling and transport and increased mortality during transport as well as induction of the PSE syndrome (poor meat quality that is pale, soft and exudative).

In many animals including warm-blooded animals, such as livestock animals, avian species and fish, stress manifests itself—directly or indirectly—in a range of forms extending from irritability to aggression or depression. Stress may lead to cardiovascular side effects ranging from slightly elevated heart rate to serious tachycardia, increased cardiovascular responsiveness and cardiac arrhythmias, which in turn can lead to sudden death. The prevalence of stress-induced lethality varies among species; some having higher stress responsiveness than others (Odoh F. M., Cadd G. G, Satterlee D. G. Genetic characterization of stress responsiveness in Japanese quail” Poult Sci. 2003, 82: 31-35).

The stress referred to here is primarily believed to be CNS-mediated stress (also called psychological stress) that in turn may cause somatic symptoms, such as changes in body temperature, cardiovascular effects, negative effects on meat quality or even mortality. Adrenergic beta-1 and adrenergic beta-2 receptor agonists at higher doses than those used for growth promotion cause direct cardiovascular effects by activating adrenergic cardiovascular beta-receptors.

The term “ractopamine” refers to the INN name for ractopamine hydrochloride or to the USAN name Ractopamine Hydrochloride or the corresponding free base.

The term “stress,” as used herein, refers to CNS-mediated (psychological) stress with consequences leading directly to the expression of psychological symptoms such as for example aggressiveness and/or indirectly to somatic symptoms or consequences as for example to effects on body temperature, circulating corticosteroids, heart rate, mortality and quality of meat products.

The term “growth promoter” as used herein, refers to a chemical entity that upon administration to livestock animals will have a favourable effect on feed efficiency and on the muscle-to-fat ratio in the carcass of said livestock animals.

The term “feed efficiency” as used herein, refers to the relationship between feed intake and weight gain in livestock animals.

The term “livestock animals” as used herein, includes animals bred for human use or consumption, including species such as bovines, ovines, porcines, caprines and equines. Without limitation to the above, the term specifically includes cattle, horses, mules, donkeys, camels, llamas, alpacas, pigs, warthogs, sheep, goats, deer, marmosets, chinchillas, rabbits and poultry such as chickens, ducks, geese, pheasants and turkeys. The term also includes fish, such as farmed fish raised in tanks, dams, rivers or sea pens.

Stress in horses can be expressed in various ways, such as for example nervousness, anxiety and tachycardia caused by administration of adrenergic agonists or by heat stress, transportation stress and feed withdrawal stress. CNS-mediated stress in horses may also lead to increased susceptibility for various diseases, such as for example allergic diseases such as heaves or infectious diseases such as staphylococcus infections. Stress in horses can be induced by drugs or aggravated by drugs, such as for example adrenergic agonists that may be given to the horses for various reasons, such as for example as bronchodilators in heaves.

Stress in pigs is very common and some pigs have been shown to carry a specific stress-gene. Pigs that are homozygous to this gene are particularly stress-prone although heterozygous pigs are also more stress-prone than pigs that do not at all express the stress-gene (Sterle J.: The Frequency of The Porcine Stress Gene in Texas Show Pigs. http://animalscience.tamu.edu.) CNS-mediated stress in pigs can be expressed in various ways, such as for example aggression, tail-biting, and tachycardia and can be caused for example by heat, transportation, stocking density, human interventions, feed withdrawal, disease and aggression between males. Stress in pigs can also be caused or aggravated by drugs, such as for example racemic ractopamine (Marchant-Forde J. N., Lay D. C., Pajor E. A., Richert B. T., Schinckel A. P.: The effects of ractopamine on the behaviour and physiology of finishing pigs. J Anim Sci. 2003, 81: 416-422). Porcine Stress Syndrome (PSS) is triggered when pigs are subjected to stress associated with transportation, restraint, fighting, mating, exercise or hot and humid weather. Pigs with PSS can become dyspneic, hyperthermic, cyanotic, develop muscle rigidity and such animals often die. Some degree of stress can be observed in most pigs and most pigs may therefore have propensity for stress. The administration of certain drugs, such as racemic ractopamine to pigs may induce or increase the symptoms of PSS in swine. In addition to the well-known fact that stress induces mortality in swine, it has been demonstrated that stress has a negative effect on the quality of meat. Thus, the muscles from stress-positive pigs often show the PSE syndrome (pale, soft and exudative). This condition causes the carcasses to be classified as being of unacceptable quality, since the meat from such animals tends to become dry when cooked. (Stadler K: Porcine Stress Syndrome and Its Effects on Maternal, Feedlot and Carcass Quantitative and Qualitative Traits. The University of Tennessee, Agricultural Extension Service, PB 1606.) The use of an adrenergic beta-agonist that does not cause stress is particularly important in animals that are already suffering from stress or have a propensity for developing stress.

Stress in cattle can be expressed in various ways, ranging from anxiety and aggression to depression, increased body temperature and increased heart rate, and can be caused by a variety of factors, such as changes in environment, transportation, human contact, aggressive herd behaviour and changes in the herd social rankings, hunger, thirst, fatigue, injury or thermal extremes (Boissy, A. & Bouissou, M-F (1995) Assessment of individual differences in behavioural reactions to heifers exposed to various fear-eliciting situations. Applied Animal Behaviour Science 46:17-31; Grandin, T (1993), Behavioural agitation during handling of cattle is persistent over time. Applied Animal Behaviour Science Vol 36:1-9). The propensity for stress in cattle seems to affect most animals and the administration of drugs, such as racemic ractopamine may induce CNS-mediated stress in cattle and particularly in cattle that are predisposed for stress. Stress in cattle can be a serious condition and may lead to decreased quality of the meat and increased lethality among the animals. The use of an adrenergic beta-agonist that does not cause stress is particularly important in animals that are already suffering from stress or have a propensity for developing stress.

As another example, sheep and goats also develop symptoms of CNS-mediated stress due to the same or similar factors as described above for other species and may include but are not limited to changes in the environment, transportation, human contact, aggressive herd behaviour, hunger, thirst, fatigue, injury or thermal extremes. The symptoms of CNS-mediated (psychological) stress are similar to those of other species and include anxiety, aggression, increased body temperature or increased heart rate. The consequences of stress are similar to those described above for other species and include risk for decreased quality of meat and sudden death of the animals. The administration of drugs, such as racemic ractopamine may induce stress in sheep and goats—particularly in predisposed animals or increase the symptoms of stress in said species. Stress in sheep and goats can be a serious condition and may lead to decreased quality of the meat and increased lethality among the animals. The use of an adrenergic beta-agonist that does not cause stress is particularly important in animals that are already suffering from stress or have a propensity for developing stress.

As still another example, such as chickens may also develop CNS-mediated stress by doses racemic ractopamine, corresponding to those necessary for obtaining increased muscle weight, decreased fat deposits and improved feed efficiency. Practically all chicken in “grower houses” are suffering from stress or are predisposed to stress because of the high stocking density (up to 20,000 birds in a very confined space). Symptoms of stress in birds, such as for example chickens, ducks, geese, turkeys, ostriches, emus and quails, can be expressed in various ways, as for example, anxiety, aggression, increased body temperature, tachycardia and lethality and can be caused for example by heat, transportation, high stocking density, sudden environmental factors, feed withdrawal, injury or disease. The administration of the beta-receptor agonist racemic ractopamine may induce or increase stress in birds. CNS-mediated stress in birds—and particularly in chicken—may lead to decreased quality of the meat and increased lethality among the animals. The use of an adrenergic beta-agonist that does not cause stress is particularly important in animals that are already suffering from stress or have a propensity for developing stress.

Stress may also manifest itself in farmed fish, such as for example barramundi, carp, cod, perch, salmon, trout and tilapia and to non-edible ornamental fish that are reared for ornamental or recreational purposes, such as for example goldfish and species of carp. Symptoms of a predisposition for stress in farmed fish can be observed as increased activity as for example during feeding frenzy and can lead to sudden death of the fish. Stress in fish can be caused by extreme temperatures, environmental factors, disease, parasites, handling or transportation for example. The administration of the exogenous beta-receptor agonist racemic ractopamine may lead to or may increase the symptoms of stress in fish, leading to decreased quality of the meat and increased lethality among the animals. The use of an adrenergic beta-agonist that does not cause stress is particularly important in animals that are already suffering from stress or have a propensity for developing stress.

Stress in pet animals, such as dogs and cats is often caused by environmental changes, such as moving to other locations, changing owners or changing feed habits. Conditions such as illnesses, travel, breeding, addition or loss of a family member or another household pet, can cause stress in animals. In such cases, pets may show symptoms of stress such as boredom, fear, anxiety and restlessness. Stressed dogs show signs of shaking, trembling, restlessness and destructiveness. The use of an adrenergic beta-agonist that does not cause stress is particularly important in animals that are already suffering from stress or have a propensity for developing stress.

Stress in animals can often be monitored by objective parameters, such as for example determination of the concentration of circulating corticosterone levels and heterophil counts. (Post J, Rebel J M J. ter Huurne A A: Physiological Effects of Elevated Plasma Corticosterone Concentrations in Broiler Chicken. An Alternate Means by which to Assess the Physiological Stress. Poultry Science, 2003. 82: 1313-1318.) Acute stress in response to exogenous adrenergic stimulation can be monitored by parameters such as body temperature, heart rate, spontaneous motility, aggression, ease of handling and even weight loss (Marchant-Forde J. N., et al. The effects of ractopamine on the behaviour and physiology of finishing pigs. J Anim Sci., 2003, 81: 410-422).

### SUMMARY OF THE INVENTION

Surprisingly, the applicant has found that parameters, such as for example the amount of adipose tissue in an animal, the amount of muscle tissue (“lean meat”) in an animal, growth of an animal, the feeding efficiency of an animal, and the muscle-to-fat ratio of an animal can be greatly improved without substantial side effects by administering to the animal a therapeutically effective amount of a ractopamine preparation wherein the preparation contains the pure or substantially pure RR enantiomer of ractopamine. The applicant has discovered that by using formulations containing a therapeutically effective amount of the pure or substantially pure RR-enantiomer of ractopamine, the beneficial effects of racemic RR/R8/SR/88 ractopamine as a growth promoter for livestock are maintained or improved, while side effects such as aggressiveness and other symptoms of stress that can be observed in animals given a formulation containing racemic RR/RS/SR/SS ractopamine can be avoided or substantially avoided by administration to said animal of a formulation containing pure or substantially pure RR-ractopamine. The term “substantially avoided” indicates that very high doses—such as doses used during toxicology testing—may still induce the side effect.

A pharmacological explanation for this surprising finding is unknown, but the major drawback with ractopamine administration, which is the drug-induced stress with concomitant negative effects on the quality of the meat as well as the increased mortality among animals treated with RR/RS/SR/SS ractopamine, can now be completely or substantially avoided. Accordingly, the invention provides in one embodiment a method of promoting or improving the growth of an animal by administering to the animal a formulation containing a therapeutically effective amount of a ractopamine preparation that contains the pure or substantially pure RR stereoisomer of ractopamine.

In a second embodiment the invention provides a method of improving the feed efficiency of an animal by administering to the animal a therapeutically effective amount of a ractopamine formulation that contains a dominance of the R stereoisomer of ractopamine. Most preferably the formulation will contain a pure or substantially pure RR-enantiomer of ractopamine.

In a third embodiment the invention provides a method of increasing the muscle to fat ratio in an animal by administering to the animal a therapeutically effective amount of a ractopamine formulation that contains a dominance of the RR stereoisomer of ractopamine. Most preferably the preparation will contain a pure or substantially pure RR-enantiomer of ractopamine.

In a fourth embodiment the invention provides an animal feed formulation including a dominance of the RR stereoisomer of ractopamine, capable of increasing lean meat deposition in an animal or for improving lean meat to fat ratio in an animal or for promoting or improving the growth of an animal or for improving the feed efficiency of an animal, mixed with a sufficient amount of animal feed to provide from about 0.1 to 500 ppm of said preparation in the feed.

In a fifth embodiment the invention provides compositions and pharmaceutical formulations for use in the above methods, which include a therapeutically effective amount of the RR stereoisomer of ractopamine. Most preferably the formulation will contain a pure or substantially pure RR-enantiomer of ractopamine.

The use of the present invention will also facilitate the handling of livestock animals, since livestock animals treated with racemic ractopamine are frequently demonstrating symptoms of stress and are therefore more difficult to handle than animals treated with RR-ractopamine.

According to the invention there are advantages of administering to livestock animals an enantiomeric mixture consisting of approximately 50% RR-ractopamine and approximately 50% SS-ractopamine, since this RR/SS enantiomeric mixture has now been found to offer the same advantages as racemic (RR-, RS-, SR′, and SS) ractopamine, but the mixture of RR- and SS-ractopamine has a lesser propensity for causing stress and stress-related side effects than a racemic mixture containing all four enantiomers of ractopamine.

There are also advantages of administering to livestock animals an enantiomeric mixture consisting of RS-ractopamine and SR-ractopamine. This enantiomeric mixture has now been found to offer the same advantages as racemic (RR-, RS-, SR-, and SS) ractopamine, but the mixture of RS- and SR-ractopamine has a lesser propensity for causing stress and stress-related side effects than a racemic mixture containing all four enantiomers of ractopamine.

Comprehension of the invention is facilitated by reading the following detailed description.

### DISCLOSURE OF THE INVENTION

General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by the specific embodiments described herein, which are intended for the purpose of exemplification only. Functionally equivalent products, formulations and methods are clearly within the scope of the invention as described herein.

The invention described herein may include one or more ranges of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

The term “animal” includes, companion animals (such as dogs and cats) as well as livestock, or farm animals such as, without limitation, cattle, pigs, sheep, goats, deer, horses, and birds (such as poultry (for example chickens, turkeys, ducks and geese)), fish and the like.

Other definitions for selected terms used herein may be found within the description of the invention and apply throughout. Unless otherwise defined, all other scientific and technical terms used herein have the same meaning as commonly understood to one of ordinary skill in the art to which the invention belongs.

### DETAILED DISCLOSURE OF THE INVENTION

The present invention relates to a method of decreasing body fat deposits, increasing muscle mass, improving feed efficiency and/or promoting or improving growth of animals, while alleviating the concomitant liability of adverse effects, development of tolerance, toxicity and side effects associated with the RR/RS/SR/SS racemic form of ractopamine. To this end, the applicant has found that by administering to an animal a therapeutically effective amount of a ractopamine formulation containing the pure or substantially pure RR-stereoisomer of ractopamine, the liability of (i.e., the amount, degree or presence of) adverse side effects can be alleviated. Moreover, by administering to animals a formulation containing the pure or substantially pure form of RR-ractopamine, side effects residing in (i.e., associated with) the RS-, SR-, and SS-enantiomers of the racemic compound can be avoided or substantially avoided.

The term “ractopamine” as used herein refers not only to the free base, but may also refer to acid addition salts and solvates thereof. Acid addition salts include, for example addition salts of ractopamine prepared with various acids, for example, hydrochloric acid, hydrobromic acid, sulphuric acid, phosphoric acid, nitric acid, or organic acids, such as citric acid, fumaric acid, tartaric acid, acetic acid, maleic acid, benzoic acid, p-toluenesulphonic acid, methanesulphonic acid, and the like.

The terms “pure RR-ractopamine”, “pure RR-enantiomer of ractopamine” and the like refer to an optical purity of RR-ractopamine that is better than 98% which means more than 98% by weight of RR-ractopamine and less than 2% of one or more of the corresponding RS-, SR- and SS-enantiomers.

The term “substantially pure RR-ractopamine” or “substantially pure RR-enantiomer of ractopamine” and the like refer to an enantiomeric purity of ractopamine wherein the % by weight of RR-ractopamine is greater than the % by weight of each of optical impurities RS-, SR- and SS-ractopamine, wherein the % by weight RR-ractopamine is sufficiently high that upon administration, adverse side effects associated with the enantiomeric impurities are ameliorated, minimised, reduced or avoided. In a more preferred embodiment, the % by weight of RR-ractopamine is at least 55%, 60% 65%, 70%, 75% or 80%. In a more preferred embodiment the enantiomeric purity of ractopamine is better than at least 90% by weight of RR-ractopamine with optical impurities consisting of 10% or less of one or more of RS-, SR- and SS-ractopamine. In the most preferred embodiment, the enantiomeric purity of ractopamine is better than at least 95% by weight of RR-ractopamine with optical impurities consisting of 5% or less of one or more of RS-, SR- and SS-ractopamine.

It is anticipated that RR-enantiomeric ractopamine will most often be used as the RR-ractopamine HC! salt, which has the USAN name butopamine hydrochloride and the chemical name RR 4-Hydroxy-a [[[3-(4-hydroxyphenyl)-1-methylpropyl]amino]methyl]benzenemethanol hydrochloride; mp 176-170.5; [alpha]_{D} −22.7°; [alpha]₃₆₅ −71.2° (c=3.7 mg/ml in methanol) (Merck Index, 1996, 12: pages 1392-1393). A “racemic mixture” of the ractopamine enantiomers can consist of a mixture of all four enantiomers or of the RR/SS enantiomeric pair or of the RS/SR enantiomeric pair.

As used herein a “therapeutically effective amount” will generally refer to an amount of RR-ractopamine that is sufficient to obtain the defined beneficial therapeutic effect. As will be realized by those skilled in the arts of pharmacology and pharmacokinetics the dose of RR-ractopamine that will constitute such an amount of drug will depend in part on the animal species, the form of administration, various pharmacokinetic parameters and the bioavailability of the form or salt of RR-ractopamine administered to said species.

The methods of the present invention are particularly useful in treating animals that already suffer from stress that is induced by their environment or by other factors, since drug-induced stress by racemic RR/RS/SR/SS ractopamine may significantly add to, or potentiate, underlying state of stress in the animals.

Preferably the RR-enantiomer of ractopamine will be used in the method of the invention, where said method is used to treat animals that are prone to stress. The use of the single enantiomeric form RR-ractopamine will reduce drug-induced toxic effects and drug-induced pharmacological side effects that reside in the RS-, SR- or SS-enantiomer of ractopamine. The use of the single enantiomeric form RR-ractopamine will completely eliminate all drug-induced toxic effects that exclusively reside in the RS-, SR or SS-enantiomer of ractopamine.

The use of the single RR-enantiomer of ractopamine, rather than the RR/RS/SR/SS racemate, as a growth promoter in livestock animals will decrease the tissue drug residues of ractopamine since RR-ractopamine is the most active of the four ractopamine enantiomers and lower total doses can therefore be used. The use of the RR-enantiomer by itself will eliminate drug residues of the other enantiomers. The use of the single RR-enantiomer rather than a racemic mixture may also have advantages at the sites of metabolism and at the receptor sites, since interactions or inhibition by the other enantiomers can be avoided when a single enantiomer is used. Furthermore, it is believed that the very potent lipolytic activity of the RR-enantiomer of ractopamine may be due to this specific enantiomer being a full agonist, meaning that this enantiomer does not cause any beta-receptor inhibition.

While it is known that racemic RR/RS/SR/SS ractopamine is causing stress in livestock animals (Marchant-Forde J. N, et al. 2003: The effects of ractopamine on the behaviour and physiology of finishing pigs” J Anim Sci, 81: 416-422), it has now been found that RR-ractopamine does not cause this side effect in livestock animals. Stress is a significant side effect of racemic RR/RS/SR/SS ractopamine and causes increased heart rate and increased mortality in animals and particularly during handling and transport as well as induction of the PSE syndrome (poor meat quality that is pale, soft and exudative) in swine. It is considered to be a major improvement of the therapy that the use of RR-ractopamine, rather than RR/RS/SR/SS ractopamine will avoid the development of stress when used in doses suitable for growth promotion in livestock animals.

Preferential selection of the RR enantiomer also offers beneficial cardiac effects in that this form of ractopamine has failed to reveal detrimental effects on cardiovascular parameters in animals at concentrations that correspond to the doses, which are suitable for growth promotion, which means doses of RR-ractopamine that decrease body fat, increase muscle mass, improve feed efficiency or increase body weight of animals. As a result of this, RR-ractopamine may be used without the significant CNS side effects of stress in livestock animals, for improving the quality of the carcass and the feed efficiency.

The method of the present invention has several advantages also for the companion animal owner or the veterinarian who wishes to increase leanness and trim unwanted fat from obese companion animals, since the present invention provides the means by which this can be accomplished. It should be noted that from the general knowledge prior to the present disclosure it would not have been expected that beta-agonist actually will decrease adipose tissues in obese animals since the lipolytic activity is usually mediated via adrenergic β-1 receptors, that will be expected to become down-regulated upon repeated administration of an adrenergic β-receptor agonist.

In one embodiment the invention resides in a method of improving or promoting the growth of an animal by administering to the animal a therapeutically effective amount of the pure or substantially pure RR ractopamine enantiomer. More particularly, the ractopamine preparation used in this form of the invention is the pure or substantially pure form of the RR-enantiomer of ractopamine. When RR-ractopamine is used as a growth promoter for livestock, it is possible to reduce the dose from the doses used for racemic ractopamine. Lowering the dose will have the added advantage of lowering the tissue residues of the drug in the carcasses of the livestock animals.

For farmers, using the method of the present invention yields leaner animals, which command higher prices from the meat industry. It was also noted that feed efficiency and/or animal growth rate are significantly enhanced when the compound of the present invention is administered to animals at therapeutically effective levels.

It may be particularly beneficial to eliminate the administration of the distomeric RS-, SR- and SS-enantiomers of ractopamine to animals since the distomeric enantiomers may cause side-effects both in the animals and in humans eating such animals particularly since at least the SS-enantiomers is completely devoid of adrenergic beta-receptor stimulating activity and the RS- and SR-enantiomers have reduced beta-receptor activity when compared with RR-ractopamine (Shallell, N. W., Feil V. J., Smith D. J., Larsen G. L., McFarland D. C.: Response of C2C12 mouse and turkey skeletal muscle cells to the beta-adrenergic agonist ractopamine. J. Anim. Sci. 2000, 78: 699-708).

Furthermore, although there usually is some variability from one animal to another and from one species to another, by administering an effective amount of only the RR-enantiomer of ractopamine, it is possible to accomplish a more “targeted” treatment. A more “targeted” treatment means that by using the RR-enantiomer, the compound's activity can be taken advantage of without also having consequences of the pharmacological side effects and the toxicological effects of the RS-, SR- or SS-enantiomers, which can be observed upon administration of a racemic mixture. This is important since it is not desirable for animals to be administered a compound with a multifaceted spectrum of activities and side effects.

In another embodiment, the invention provides a method of improving the feed efficiency of an animal by administering to the animal a therapeutically effective amount of a ractopamine preparation devoid or substantially devoid of the RS-, SR- and SS-enantiomers of ractopamine. More particularly, the ractopamine preparation used in this form of the invention is a pure or substantially pure form of the RR-enantiomer of ractopamine.

In still another embodiment, the invention provides a method of increasing the muscle to fat ratio in an animal by administering to the animal a therapeutically effective amount of a ractopamine preparation devoid or substantially devoid of at least the SS enantiomer of ractopamine. More particularly, the ractopamine preparation used in this form of the invention is a pure or substantially pure form of the RR-enantiomer of ractopamine.

In a further embodiment, the invention provides methods for prophylactic treatment to individuals with hereditary or environmental risks for the development of obesity. The method also provides method for treating atrophied muscles, such as for example after prolonged inactivity or prolonged limited activity or due to rapid loss of weight caused by extremely restrictive diets that severely restrict calorific intake. Thus the present invention provides both symptomatic and prophylactic treatment for animals and humans in need of improved or increased muscle mass. According to this embodiment, the present invention provides a method of use for reducing fat deposits in animals and humans and in particular in obese animals, including obese companion animals and obese humans by administering to the subject in need thereof an effective amount of a ractopamine preparation containing the pure or substantially pure RR-enantiomer of ractopamine. More particularly, the ractopamine preparation used in this form of the invention may preferentially contain the pure or substantially pure RR-enantiomer of ractopamine, although racemic ractopamine has now been found to cause weight loss in obese animals.

Reduction in fat deposit is known to improve health in all obese mammals. The invention may also provide prophylactic treatment to individuals with hereditary or environmental risks for the development of obesity. Thus the present invention provides both symptomatic and prophylactic treatment for animals and humans suffering from obesity or at risk for developing obesity. Use of the methods of the present invention provide a means for decreasing drug residues in livestock animals, since animals administered the pure and single RR-ractopamine enantiomer are administered less drug than animals given a racemic mixture of ractopamine enantiomers. There are also additional pharmacokinetic advantages by using the pure or substantially pure RR-enantiomer of ractopamine, relating to the metabolic and excretion rates of the ractopamine enantiomers.

It is of particular importance to reduce or eliminate the use of the RS-, SR and SS-enantiomers of ractopamine since these enantiomers do not carry any or only limited growth promoting activity, but carry unwanted activities, such as for example risk for stress and concomitant decrease in meat quality and concomitant risk for lethality. There is a risk that pharmacological side effects and toxic effects of the RS-, SR- and SS-enantiomers of racemic ractopamine can be expressed in humans who have been eating meat from animals that have been administered racemic ractopamine, particularly if the racemic form of ractopamine is given in high doses to livestock animals. There may also be risks for unborn children and lactating infants when the mother eats meat treated with a mixture of ractopamine enantiomers, such risk being reduced or eliminated if the livestock animals are treated with the single RR-enantiomer of ractopamine rather than a racemic mixture. Thus, the present invention is reducing the risk for deleterious pharmacological side effects and toxic effects in humans, such side effects being hereinbefore described and originating from drug residues in animals, having been treated with racemic ractopamine.

The use of the single RR-enantiomer of ractopamine in animal species, such as livestock species, and in humans, eliminates any side effect that is the result of interaction by the SS-, RS- or SR-enantiomers of ractopamine with the absorption, distribution, metabolism and excretion of RR-ractopamine.

Use of the methods of the present invention provides a means for improving the quality of meat from livestock animals by reducing stress and factors leading to stress, said stress being known to decrease the quality of meat (Sterle J.: The Frequency of the Porcine Stress Gene in Texas Show Pigs. http://animalscience.tamu.edu.; Chatillon G. (1994): “Transport mortality has its origin in stress: how to get pigs to their destination in good condition. Porc. Magazine 265: 37-41; Warris P. D.; Brown S. N. and Adams S. J. M. 1994: “Relationship between subjective and objective assessment of stress a slaughter and meat quality in pigs. Meat Science 38:329-340).

Use of the methods of the present invention also provides a means of preventing or reducing morbidity, particularly attributable to stress, stress during transportation, aggressive interactions between animals and cardiovascular or respiratory events caused directly or indirectly as a consequence of the administration of the SS, RR, or SR enantiomers of ractopamine to the animals.

The environmental impact of dosing livestock animals with RR-ractopamine rather than racemic ractopamine will be significant since neither the RS-, SR and SS-enantiomers of ractopamine nor the metabolites thereof will pollute the environment. Additionally, a favourable environmental impact will come from the fact that the doses of RR-ractopamine by weight will usually be lower than the corresponding doses of racemic ractopamine.

The present invention also relates to food compositions including an admixture of food materials with the RR-enantiomer of ractopamine, the RR-enantiomer of ractopamine being substantially free of the SS-, RS- and SR-enantiomers. RR-ractopamine is preferentially administered to animals that are being given a diet, consisting of protein-containing food materials in order to build muscle mass in said animals. Accordingly in another embodiment the invention provides an animal feed preparation containing pure or substantially pure RR-ractopamine and being capable of increasing lean meat (muscle) content in an animal or improving the muscle-to-fat ratio in an animal or improving the growth of an animal or improving the feed efficiency of an animal, while avoiding certain side effects, such as for example stress, comprising a therapeutically effective amount of a ractopamine preparation containing a pure or substantially pure RR-enantiomer of ractopamine. Animal feed containing RR-ractopamine will contain the pure or substantially pure active RR-enantiomer of ractopamine mixed with a sufficient amount of animal feed to provide from about 1 to 500 ppm of RR-ractopamine in the feed.

Animal feed supplements can be prepared by admixing about 75% to 95% by weight of a ractopamine preparation as herein described with about 5% to 25% by weight of a suitable carrier or diluent. Carriers suitable for use to make up the feed supplement compositions include the following: alfalfa meal, soybean meal, cottonseed oil meal, linseed oil meal, sodium chloride, cornmeal, cane molasses, urea, bone meal, corncob meal, rice kernel and the like. The carrier promotes a uniform distribution of the active ingredients in the finished feed into which the supplement is blended. It thus performs an important function by ensuring proper distribution of the active ingredient throughout the feed and supplying the animal with the protein that is a prerequisite for muscle growth in all species

If the supplement is used as a top dressing for feed, the carrier likewise helps to ensure uniformity of distribution of the active material across the top of the dressed feed.

The preferred medicated swine feed generally contain from 1 to 200 grams of 10 pure or substantially pure RR-ractopamine per ton of feed, the optimum amount for these animals usually being about 1 to 100 grams per ton of feed.

The preferred medicated drinking water for swine will generally contain 1 to 200 ppm by weight of RR-ractopamine.

The preferred medicated cattle, sheep and goat feed generally contain from 5 to 500 grams of pure or substantially pure RR-ractopamine per ton of feed, the optimum amount for these animals usually being about 10 to 200 grams per ton of feed.

The preferred medicated drinking water for cattle, sheep and goats generally contain 5 to 500 ppm by weight of RR-ractopamine.

The preferred medicated poultry and turkey feed generally contain from 1 to 100 grams of pure or substantially pure RR-ractopamine per ton of feed, the optimum amount for these animals usually being about 2 to 50 grams per ton of feed.

The preferred medicated drinking water for poultry and turkeys generally contain 1 to 50 ppm by weight.

The preferred medicated dogs and cats feed generally contain from 1 to 100 grams of pure or substantially pure RR-ractopamine per ton of feed, the optimum amount for these animals usually being about 2 to 50 grams per ton of feed.

The preferred medicated drinking water for dogs and cats generally contain 1 to 100 ppm by weight.

It was surprisingly found that solutions containing water-soluble salt forms of racemic RR/RS/SR/SS ractopamine in the drinking water are uniquely suitable for administration to livestock animals, including chicken and to horses and pet animals. The concentrations of a water-soluble salt form of racemic RR/RS/SR/SS ractopamine in the drinking water are within the limits stated above for medicated drinking water containing RR-ractopamine for swine, cattle, sheep, goats, horses, chicken, turkeys, dogs and cats.

It has also been found that racemic ractopamine is well suited for administration in implanted reservoirs, as for example reservoirs to be implanted into the rumen of cattle or sheep, as described in for example U.S. Pat. Nos. 6,855,334 and 6,974,587.

It was surprisingly found that racemic RR/SS ractopamine has advantages over a racemic mixture containing all four RR, RS, SR and SS enantiomers. Thus, a mixture of the RR- and SS-enantiomers, containing approximately 50% of each of the enantiomers, is chemically stable and has metabolic and pharmacokinetic advantages, and the RR/SS racemate is also devoid of any side effects that are exclusively residing in the RS- and the SR-enantiomers. The racemic mixture RR/SS is also more potent as a beta-receptor agonist than the RR/RS/SR/SS racemate. The RR/SS mixture may therefore be used in doses that are lower than a corresponding mixture containing all four RR, RS, SR and SS enantiomers. The environmental impact of the RR/SS mixture is considerable less than the impact by a racemate containing all four enantiomers. The concentrations of a RS/SR ractopamine in the feed of various animal species are within the frames for each species as stated above for RR-ractopamine. Water-soluble salt form of racemic RR/SS ractopamine in the drinking water are within the limits for RR-ractopamine, stated above for medicated drinking water for swine, cattle, sheep, goats, horses, chicken, turkeys, dogs and cats.

In another embodiment, the invention provides compositions and pharmaceutical formulations for the use of the RR/SS racemic mixture as a growth promoter for livestock, which include a therapeutically effective amount of the pure or substantially pure racemic mixture RR/SS-ractopamine. The formulations to be used for the RR/SS racemic mixture are identical to the feed and drinking water for RR-ractopamine, described herein, wherein RR/SS-ractopamine is used instead of RR-ractopamine.

The terms “pure RR/SS-ractopamine”, “pure RR/SS-racemate of ractopamine” and the like refer to an enantiomeric purity of RR/SS-ractopamine that is better than 98%, which means more than 98% of racemic RR/SS-ractopamine and less than 2% of one or both of the RS- and SR-enantiomers.

The term “substantially pure RR/SS-ractopamine” and the like refer to an enantiomeric purity of ractopamine wherein the combined % by weight of RR-ractopamine and SS-ractopamine is greater than the % by weight of each of RS- and SR-ractopamine. In a more preferred embodiment, the combined % by weight of RR-ractopamine and SS-ractopamine is at least 55%, 60%, 65%, 70%, 75% or 80%. In a more preferred embodiment the enantiomeric purity of RR/SS-ractopamine is better than at least 90% by weight of RR/SS-ractopamine with enantiomeric impurities consisting of 10% or less of one or both of the RS- and SR-enantiomers of ractopamine. In the most preferred embodiment, the enantiomeric purity of RR/SS-ractopamine is better than at least 95% by weight of RR/SS-ractopamine with enantiomeric impurities consisting of 5% or less of one or both of the RS- and SR-enantiomers of ractopamine.

In still another embodiment the invention provides compositions and pharmaceutical preparations for use in above methods, which include a therapeutically effective amount of the pure or substantially pure racemate RR/SS ractopamine. Those skilled in the art of chiral pharmacology realize that a eutectic mixture of the RR and SS-enantiomers probably exists and can be expected to have certain pharmacological advantages over racemic mixtures.

It was surprisingly found that racemic mixture of ractopamine containing the two enantiomers RS and SR only has advantages over a racemic mixture containing all four RR, RS, SR and SS enantiomers. Thus, a mixture of the RS- and SR-enantiomers, containing approximately 50% of each of the enantiomers, has metabolic and pharmacokinetic advantages, and the RS/SR racemate is also devoid of any side effects that are residing in the RR- and the SS-enantiomers. Thus, the racemic mixture RS/SR is expressing stress in the animals to a significantly lesser degree than the racemic mixture containing all four enantiomers. The concentrations of a RR/SR ractopamine in the feed of various animal species are within the frames for each species as stated above for RR-ractopamine. The environmental impact of the RR/SR mixture is less than the impact by a racemate containing all four enantiomers. The concentration of a water-soluble salt form of racemic RS/SR ractopamine in the drinking water is within the limits stated above for medicated drinking water for swine, cattle, sheep, goats, horses, chicken, turkeys, dogs and cats. In another embodiment, the invention provides compositions and pharmaceutical formulations for the use of the RS/SR racemic mixture as a growth promoter for livestock, which include a therapeutically effective amount of the pure or substantially pure racemic mixture RS/SR ractopamine. The formulations to be used for the RS/SR racemic mixture are identical to the feed and drinking water for RR-ractopamine, described herein, wherein RS/SR-ractopamine is used instead of RR-ractopamine. Those skilled in the art of chiral pharmacology realize that a eutectic mixture of the RS and SR-enantiomers probably exists and can be expected to have certain pharmacological advantages over racemic mixtures.

The terms “pure RS/SR ractopamine” pure RS/SR-racemate of ractopamine” and the like refer to an enantiomeric purity of RS/SR ractopamine that is better than 98% which means more than 98% of racemic RS/SR-ractopamine and less than 2% or less of one or both of the RR- and SS-enantiomers of ractopamine.

The term “substantially pure RS/SR-ractopamine” and the like refer to an enantiomeric purity of ractopamine wherein the combined % by weight of RS-ractopamine and SR-ractopamine is greater than the % by weight of each of RR- and SS-ractopamine. In a more preferred embodiment, the combined % by weight of RS-ractopamine and SR-ractopamine is at least 55%, 60%. 65%, 70%. 75% or 80%. In a more preferred embodiment the enantiomeric purity of RS/SR-ractopamine is better than at least 90% by weight of RS/SR-ractopamine with enantiomeric impurities consisting of 10% or less of one or both of the RR- and SS-enantiomers of ractopamine. In the most preferred embodiment, the enantiomeric purity of RS/SR-ractopamine is better than at least 95% by weight of RS/SR-ractopamine with enantiomeric impurities consisting of 5% or less of one or both of the RR- and SS-enantiomers of ractopamine.

In still another embodiment the invention provides compositions and pharmaceutical preparations for use in above methods, which include a therapeutically effective amount of the pure or substantially pure racemate RS/SR ractopamine. Those skilled in the art of chiral pharmacology realize that a eutectic mixture of the RS and SR-enantiomers may exist and can be expected to have certain pharmacological advantages over racemic mixtures.

In the present method, the ractopamine preparations can be administered by any suitable means, including parenterally, transdermally, subcutaneously, intravenously, intramuscularly or orally, topically, nasally, rectally, by inhalation or via implanted reservoirs or pellets containing the drug. The form in which the drug will be administered (e.g. injectable, inhalant, powder, granulate, tablet, capsule, solution, emulsion, subcutaneous pellet, transdermal patch, suppositories, sprays, aerosols or reservoirs to be implanted into the rumen of cattle or sheep, etc.) will depend on the route by which it is administered.

The preferred route of administration is the oral route, with the eutomer of ractopamine mixed into the feed or the drinking water of livestock animals. RR-ractopamine may be administered in tablets, capsules, caplets, solutions, subcutaneous pellets, transdermal patches or a similar form to non-food animals.

Formulations for oral use include tablets, which contain the active ingredient in admixture with non-toxic pharmaceutically acceptable excipients. These excipients may be, for example, inert diluents, such as calcium carbonate, sodium chloride, lactose, calcium phosphate or sodium phosphate; granulating and disintegrating agents, for example, potato starch or alginic acid; binding agents, for example, starch, gelatine or acacia; and lubricating agents, for example, magnesium stearate, stearic acid or talc. Other pharmaceutically acceptable excipients can be colorants, flavouring agents, plasticizers, humectants etc. The tablets may be uncoated or they may be coated by known techniques, optionally to delay disintegration and absorption in the gastrointestinal tract and thereby provide a sustained action over a longer period (i.e. controlled release). For example, a time delay material such as glyceryl monostearate or glyceryl distearate may be employed.

Formulations for oral use of RR-ractopamine or any of the racemates described herein may also be presented as chewing tablets, or as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil.

Formulations of RR-ractopamine or any of the racemates described herein may also be presented as a more robust form of dog and or cat treats where the formulation for oral administration may be presented as a chewable formulation consisting of a vegetable or cereal matrix for example and a combination of minerals and vitamins which will be well known to those skilled in the art together with other pharmaceutically acceptable excipients such as colorants, flavouring agents, plasticizers, humectants, palatability enhancers etc. In such formulations, the active ingredient is mixed with water or an oil medium, for example, peanut oil, liquid paraffin, or olive oil.

Powders, dispersible powders or granules suitable for preparation of an aqueous suspension by addition of water are also convenient dosage forms of the present invention. Formulation as a suspension provides the active ingredient in admixture with a dispersing or wetting agent, suspending agent and one or more preservatives. Suitable dispersing or wetting agents are, for example, naturally-occurring phosphatides, as e.g. lecithin, or condensation products of ethylene oxide with e.g. a fatty acid, a long chain aliphatic alcohol or a partial ester derived from fatty acids and a hexitol or a hexitol anhydrides, for example, polyoxyethylene stearate, polyoxyethylene sorbitol monooleate, polyoxyethylene sorbitan monooleate etc. Suitable suspending agents are, for example, sodium carboxymethylcellulose, methylcellulose, sodium alginate etc.

Additionally, other preferred forms of administration are by inhalation or by transdermal delivery systems or subcutaneous delivery systems, which will reduce or avoid gastrointestinal metabolism and hepatic first-pass metabolism by metabolizing enzymes; such delivery systems may be designed to prolong the absorption or decrease the peak plasma drug concentration (Cmax) or to increase the exposure of the animal to the drug (increased AUC, meaning Area Under a Curve where plasma drug concentration has been plotted over time).

Preparations of RR-ractopamine or any of the racemates described herein may also be administered parenterally (intravenous, intramuscular, subcutaneous or the like) in dosage forms or formulations containing conventional, non-toxic pharmaceutically acceptable carriers and adjuvants. The formulation and preparation of such compositions is well-known to those skilled in the art of pharmaceutical formulation. Additional information can be obtained in medical and pharmaceutical textbooks, such as for example Goodman & Gilman: The Pharmacological Basis of Therapeutics. Section 1. McGraw-Hill, Ed 9, ISBN 0-07-026266-7. For parenteral use, the pharmaceutical compositions according to the invention may comprise the preparation in the form of a sterile injection. To prepare such a composition, the preparation is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate.

For parenteral administration ractopamine preparations may be prepared in the form of a paste or pellet and administered as an implant, usually under the skin of the head or ear of the animal in which increase in lean meat deposition and improvement in lean meat to fat ratio is sought.

As an alternative to a paste, pellet or subcutaneous implant, parenteral administration may involve injection of a solution, containing sufficient amount of the pure or substantially pure RR-ractopamine to provide the animal with 0.1 to 100 mg/day of the active ingredient. Preferred parenteral dosages for swine, cattle, sheep and goats are in the range of from 0.5 to 100 mg/day of pure or substantially pure RR-ractopamine; whereas, the preferred dose level of said parenteral ractopamine preparation for poultry and turkeys are in the range of from 0.05 to 20 mg/day. Formulations containing RR/SS ractopamine or RS/SR ractopamine will use up to twice as much of the racemic ingredients as compared to formulations containing the pure or substantially pure RR-enantiomer of ractopamine.

For the rectal application, suitable dosage forms for a composition according to the present invention include suppositories (emulsion or suspension type), and rectal gelatin capsules (solutions or suspensions). In a typical suppository formulation, the ractopamine preparation is combined with an appropriate pharmaceutically acceptable suppository base such as cocoa butter, esterified fatty acids, glycerinated gelatin, and various water-soluble or dispersible bases like polyethylene glycols and polyoxyethylene sorbitan fatty acid esters. Various additives like e.g. enhancers or surfactants may be incorporated.

For the nasal application, typical dosage forms for a composition according to the present invention include nasal sprays and aerosols. In a typically nasal formulation, the active ingredients are dissolved or dispersed in a suitable vehicle. The pharmaceutically acceptable vehicles and excipients and optionally other pharmaceutically acceptable materials present in the composition such as diluents, enhances, flavouring agents, preservatives etc. are all selected in accordance with conventional pharmaceutical practice in a manner understood by the persons skilled in the art of formulating pharmaceuticals.

The ractopamine preparations according to the invention may also be administered topically on the skin for percutaneous absorption in dosage forms or formulations containing conventionally non-toxic pharmaceutically acceptable carriers and excipients that may include microspheres and liposomes. The ractopamine preparations include creams, ointments, lotions, liniments, gels, hydrogels, solutions, suspensions, pastes, plasters and other kinds of transdermal drug delivery systems. The ractopamine preparations may include emulsifying agents, antioxidants, buffering agents, preservatives, humectants, penetration enhancers, chelating agents, gel forming agents, ointment bases, perfumes and skin protective agents.

The quantity of the drug to be administered to an animal will have to be titrated for each species. In general, the doses of the RR-ractopamine preparation to be used in swine may—as an example—be about 5 ppm of RR-ractopamine included in the feed for swine. With a total treatment period of 28 days and 50 mg/head/day, this corresponds to a total consumption of 1.4 grams of RR-ractopamine for each swine. It is usually calculated that finishing swine have a feed consumption of 3 kg/day. The doses of the RR-ractopamine preparation to be used in cattle may—as an example—be about 20 ppm of RR-ractopamine in feed for cattle and a treatment period of 28 days and 200 mg/head/day, this corresponds to a total consumption of 5.6 grams of RR-ractopamine for each cow. It is usually calculated that finishing cattle have a feed consumption of 10 kg/day. The total consumption of RR-ractopamine will be decreased or increased with changes in the feed inclusion of RR-ractopamine and with changes in the duration of the treatment period. The daily dose of RR-ractopamine to cattle may be as high as 500 mg/head/day. It is common and it may be found advantageous to change the feed inclusion of growth promotants like RR-ractopamine during the treatment period and all changes in the doses administered to the animals will of course influence the total consumption per animal of active ingredient.

It may be advantageous to administer RR-ractopamine as an implantable subcutaneous controlled-release pellet, designed to deliver from 1 mg/day to 300 mg/day for the entire treatment period, which may last up to six or eight weeks, whereupon the animal may be slaughtered without any withholding period (drug-free days before slaughter) or with a short withholding period of one to three days. For all livestock species, the doses of RR-ractopamine have to be carefully titrated and will depend on the pharmacological efficacy of the drug in the selected species or sub-species, the metabolic fate and rate of excretion of the drug in various species, the route of administration, the size of the mammal or bird and the results sought. In general, quantities of growth promotant sufficient to decrease body fat, increase muscle mass, and improve feed efficiency will be administered. The actual dosage (quantity administered at a time) and the number of administrations per day will depend on the pharmacokinetic property of the drug and the metabolism of the drug in the body of the specific mammal species. For example about 10 to 3000 micrograms of the pure RR-enantiomer of ractopamine may be given by various forms of inhalation devices, such as metered dose inhalers and nebulizers, 0.01 to 500 milligrams may be given by the oral route (for example as powders, granulates, tablets or liquids) one to four times per day (or as ad lib daily doses to animals) and may be an adequate dose in most livestock animals to produce the desired effect. Suitable oral doses in humans include doses in the range. of a dose of 0.05 mg to 5 mg once daily or said doses given repeatedly up to six times during the day. The actual and finally titrated drug doses may be higher or lower and administration may take place more or less frequently than indicated above, as determined by clinical studies or by the caring individual, physician or veterinarian.

It is, of course, recognized that more than one pellet may be administered to an animal to achieve the desired dose level which will provide the increase in lean meat disposition and improvement in lean meat to fat ratio desired. Moreover, it has been found that implants may also be made periodically during the animal treatment period in order to maintain the proper drug level in the animal's body.

The pharmacological side effects of RR-ractopamine in high dose include tachycardia, tremor and possibly CNS-mediated effects. These and other side effects may be of short duration and may be associated with peak plasma concentrations of the drug in connection with a drug overdose. These side effects can be reduced or completely avoided by using drug delivery systems that slowly release the drug of the present invention into the systemic circulation. Such slow-release or controlled-release delivery systems include granulae, tablets, capsules, subcutaneous pellets or forms of reservoir depots with slow-release or controlled-release properties that are designed to release the active ingredient slowly or in a controlled manner.

In the method of the present invention, the RR-enantiomer of ractopamine or either of the RR/SS or the RS/SR racemates of ractopamine can be administered together with one or more other active compound(s). Compounds that improve or prolong the therapeutic effect of beta-agonists, e.g. compounds that delay or inhibit the absorption or the metabolic degradation of the compound, may also be co-administered with the eutomeric beta agonist to further improve the therapeutic activity. Other drugs such as for example other growth promoting agents and antibacterial compounds may be combined with the selected drug of the present invention to obtain improved health of the animal or improved growth promoting activity of the formulation. Recombinant porcine somatotropin or similar compounds may be used in combination with the present invention to further enhance the effect of the adrenergic beta-agonist of the present invention; Hansen J. A., Yen J. T., Nelssen J. L., Nienaber J. A., Goodband R. D., Wheeler T. L., 1997: Effects of somatotropin and salbutamol in three genotypes of finishing barrows: growth, carcass and calorimeter criteria. J. Anim. Sci. 75: 1798-1809.

Antiviral and antibacterial agents, including antibiotics, may be used together with compounds of the present invention in order to prevent or control viral or bacterial infections or to improve on antiviral or antibacterial effects of the compounds of the present invention. Antiviral and antibacterial compounds may also have growth promoter activity. Thus, antiviral and antibacterial agents may increase or promote or potentiate the effects of the compounds of the present invention on fat reduction, muscle growth or feed efficiency.

Eutomeric mixtures of RR-ractopamine and another active component or other active components may be used and may have certain advantages, such as for example leanliness (muscle/fat), pharmacokinetic or metabolic advantages or may have improved side effects or improved receptor activity as realized by those skilled in the art of chiral drug pharmacology. Eutectic mixtures of RR-ractopamine and SS-ractopamine or of RS-ractopamine and SR-ractopamine can also be expected to have certain advantages, such as for example improved receptor activity in tests performed in vitro.

RR-ractopamine can be used for weight loss therapy and will cause loss of fat tissue in obese animals and humans. When used for weight-loss purposes, the administration of RR-ractopamine should preferably be combined with appropriate life-style modifications, such as for example modified eating habits and increased exercise.

Chemistry

Ractopamine stereoisomers can be synthesized according to the methodology by Ricke et al 1999 which is hereby included by reference in its entirety (Ricke E A, Smith D J, Fell V J, Larsen C L, Caton J S Effects of ractopamine hydrochloride on stereoisomers on growth, nitrogen retention and carcass composition in rats. J. Anim, Sci. 1999 77:701-707).

The stereoisomeric compound to be used may be separated from the other stereoisomers by means of techniques known per se, for example, selective crystallization of an addition salt with a stereoisomer of an acid with a chiral center. However, a stereochemically pure starting substance or a stereochemically pure intermediate product obtained by isomer separation is preferably used. This substance may then be converted in one or more reaction steps into the desired stereochemically pure compound, for example, as described in the paper by Van Dijk and coworkers mentioned hereinafter.

These and other advantages will become apparent from the examples set forth below. Such examples are provided only for exemplification of the invention and are not to be considered as limiting the invention.

Racemic ractopamine can be made by methods that are obvious to those skilled in the art of chemistry. A synthetic method was described by van Dijk J. and Moed H. D. in Recueil des Traveaux Chimiques, 1973, Vol. 92: 1281-1297 and is hereby included in its entirety by reference.

Racemic ractopamine can also be isolated and purified from conveniently available commercial sources. One kilogram of racemic ractopamine was stirred overnight with 15 L of water. The mass was filtered, and the filtrate (11.5 L) was evaporated under reduced pressure to a small volume (ca. 1-2 L). Aqueous potassium carbonate was added to raise pH to ca. 10, and the solution was extracted twice with equal volumes of ethyl acetate. The ethyl acetate extracts were combined, evaporated under reduced pressure to ca. 0.5 L, and an equal volume of hexanes added. After standing overnight, crystals of ractopamine free base (6.83 g) were filtrated. This material was crystallized again from ethyl acetate/hexanes to give pure ractopamine free base (6.01 g). HPLC showed 98.5% purity. A sample of ractopamine free base (120 mg) was stirred with 10 ml of deionized water cooled in an ice-water bath, and 2 M aqueous hydrochloric acid (2 ml) was added slowly. After stirring to dissolve the material, the solution was filtered and lyophilized to give ractopamine hydrochloride (125 mg). The mp was 128-135° C., lit (Merck Index) mp 124-129° C. ¹H NMR was consistent. HPLC showed 98.0% purity.

The stereoisomeric compound RR-ractopamine can be isolated from the other stereoisomers by means of techniques known per se, for example, selective crystallization of an addition salt with a stereoisomer of an acid with a chiral center. However, a stereochemically pure starting substance or a stereochemically pure intermediate product obtained by isomer separation is preferably used. This substance may then be converted in one or more reaction steps into the desired stereochemically pure compound, for example, as described in the paper by Van Dijk and coworkers mentioned hereinafter.

The RR-enantiomer of ractopamine can also be obtained by stereoselective synthesis or by resolution of a racemic mixture of enantiomers.

Methods for the preparation of RR-ractopamine have been described by Mills J. et al. in Eur. Pat Appln 7,205 and by Anderson D. B. et al. in U.S. Pat. No. 4,690,951, both of which are hereby incorporated in their entirety by reference. Other standard methods of resolution known to those skilled in the art including but not limited to crystallization and chromatographic resolution and can also be used. (See for example, Stereochemistry of Carbon Compounds, E. L. Eliel, McGraw Hill 1962; “Tables of Resolving Agents,” S. A. Wilen and Lochmuller, L. H. et al., 1975, J. Chromatogr. 113(3): 283-302.) Additionally, the optically pure (RR) isomer can be prepared from the racemic mixture by enzymatic biocatalytic resolution. See, for example, U.S. Pat. Nos. 5,057,427 and 5,077,217, the disclosures of which are incorporated herein by reference. Additionally, the 30 preparation of RR-enantiomeric phenethanolamines was described by Anderson D. B. et al.: Growth promotion, U.S. Pat. No. 5,643,967, which is hereby included by reference in its entirety.

Biological Effects

The growth promoting activity of the adrenergic beta-receptor agonist ractopamine has been demonstrated in various livestock species (Watkins L. E.; Joens D. H.; Mowrey D. H.; Anderson D. B. and Veenhuizen L. 1990: “The effect of various levels of ractopamine hydrochloride on the performance and carcass characteristics of finishing swine.” J. Anim. Sci. 68: 3588-3595; Williams N. H.; Cline T. R.; Schinkel A. P. and Jones D. J. 1994: “The impact of ractopamine, energy intake and dietary fat on finisher pig growth performance and carcass merit.” J. Anim. Sci. 72: 3152-3162; Mills S. E.: 2001: “Biological Basis of Ractopamine Response.” J. Anim. Sci. 79 (Suppl. 1): E28-32.)

To those skilled in the art of pharmacology, it is known that synthetic adrenergic beta-receptor agonists have numerous effects, that may have similarities to endogenous adrenergic beta-receptor agonists, of which adrenaline and noradrenaline are the most well known.

There are three types of adrenergic beta-receptors: Stimulation of beta-1 receptors leads—for example—to increased heart rate, increased cardiac contractility and increased blood pressure. Stimulation of adrenergic beta-2 receptors leads—for example—to relaxation of various types of smooth muscles, such as bronchial smooth muscle. Adrenergic beta-3-adrenergic receptors are involved in the regulation of lipolysis and thermogenesis.

Adipose tissue has beta-1, beta-2 and beta-3 receptors and stimulation of these receptors lead to lipolysis, which means that fat molecules are broken down. Stimulation of beta-receptors has also been shown to inhibit lipogenesis, which means that stimulation of these receptors can inhibit the formation of new fat cells. However, it is believed that the lipolytic activity by adrenergic beta-receptor agonists is more important than the effects of beta-agonists on the formation of new fat cells. Adrenergic beta-receptor stimulation is also known to increase muscle mass by a mechanism that is believed to involve inhibition of protein breakdown in the continuously ongoing process of formation and degradation of muscle proteins (Bardsley R G, Allcock S M J, Dawson J M, Dumelow N W, Higgins J A. Lasslett Y V, Lockey A K, Buttery P J Effect of β-agonists on expression of calpain and calpastatin activity in skeletal muscle. Biochimie, 1992, 74:267-273)

Thus, stimulation of adrenergic beta-2 receptors at various locations in the body will lead to decreased fat deposits and increased muscle mass, and the major side effect consists of relaxation of bronchial smooth muscle, which is not at all a serious side effect. While stimulation of adrenergic beta-1 receptors causes a similar breakdown of fat and a similar increase of muscle proteins as stimulation of beta-2 receptors, there can be serious cardiovascular side effects of beta-1 stimulation.

Neither racemic RR/RS/SS ractopamine nor any of the ractopamine enantiomers have high affinity for adrenergic beta-3 receptors.

Thus—although there is a significant overlap between the effects of beta-1 and beta-2 receptor stimulation, activation of beta-2 receptors is considered to be the most advantageous receptor-type in animals as repartitioning agents. Beta-1 stimulation is less advantageous because of an increased risk for cardiovascular side effects that may include tachycardia and cardiac arrhythmias.

It has now been found that racemic RR/RS/SR/SS-ractopamine has selectivity for adrenergic β-1 receptors (see above under the heading “BACKGROUND TO THE INVENTION”) It has also been found that the RR-enantiomer is mainly not a selective β-1 receptor agonist, but RR-ractopamine has actually higher affinity for receptors than for β-1 receptors.

Those skilled in the art of pharmacology avoid linking in vivo activity of adrenergic beta-agonists directly to receptor affinity of drugs, since the ultimate effects in vivo depend not only on receptor affinity, but also on the size of the available receptor population in various organs. The complicated situation in various organs can be exemplified with the availability of β-1 and β-2 receptors in the human heart, where, under normal circumstances, the distribution of adrenergic beta-receptors are approximately 77% β-1 receptors+23% β-2 receptors. Adrenergic receptor populations can be changed under pathological circumstances and during heart failure the human heart has less β-1 receptors than normal, and as a result the beta-receptor population in the failing heart consists of about 60% β-1 receptors+about 40% β-2 receptors (Bristow M R, Ginsburg R, Umans V, Fowler M, Minobe W, Rasmussen R, Zera P, Menlove R, Shah P, Jamieson S, Stinson E. β-1 and β-2 adrenergic receptor subpopulations in non-failing and failing human ventricular myocardium. (Circ Res 1986, 59: 297-309.) Adrenergic beta-receptors may also be down-regulated in selected organs upon repeated stimulation with an adrenergic agonist. The term “down-regulated” refers to the fact that individual receptors seem to disappear from the cell membrane by internalization into the cell. As another example of the complexity, the concentration of adrenergic beta-receptors in adipose tissue can be reduced by 50% by exposing animals to racemic ractopamine. (Spurlock M E, Cusumano J C, Ji Q, Anderson D B, Smith-II C K, Hancock D L, Mills S E. The effect of ractopamine on β-adrenoceptor density and affinity in porcine adipose and skeletal muscle tissue. J. Anim, Sci. 1994, 72:75-80.)

The following studies have been initiated by us and are performed in laboratory animals under our close supervision to study CNS side effects of RR-ractopamine and racemic mixtures of ractopamine:

Ongoing neuropharmacological studies in rats (Irwin test) being administered the test articles orally, demonstrate stress by racemic RR/RS/SR/SS ractopamine, thereby supporting the findings of Marchant-Forde et al (Marchant-Forde J. N., et. al. 2003: “The effects of ractopamine on the behaviour and physiology of finishing pigs” J Anim Sci., 81: 416-422), but surprisingly do not show signs of drug-related stress by the pure RR-enantiomer of ractopamine. The reference compound R-salbutamol does not induce stress in pigs (London C. J., Aberg G., Sadler M., Marchant-Forde J. N.: Effects of a New Growth Promoter (R-albuterol) for Commercial Swine Production. Abstr. Bio 2005, Philadelphia, US. June 2005) and is not causing CNS-effects related to stress in laboratory animals.

Preliminary results from stress testing of RR-ractopamine, RR/RS/SR/SS-ractopamine and R-salbutamol in mice using the methodology described by Aberg (U.S. Pat. No. 6,372,799) demonstrate CNS-mediated stress in animals treated with racemic ractopamine, while surprisingly, RR-ractopamine is not causing stress.

Spontaneous motor activity studies in mice being administered test articles orally, demonstrates drug-related and CNS-mediated (“psychological”) stress by racemic RR/RS/SR/SS-ractopamine, but surprisingly, there were no signs of drug-related stress by the pure RR-enantiomer of ractopamine.

Conclusions from current tests in animals:

It can be concluded from studies in animals, that RR/RS/SR/SS-ractopamine is causing drug-related symptoms that are indicative of CNS-mediated stress, while it has surprisingly been found the pure RR-enantiomer of ractopamine does not cause CNS-mediated stress in animals.

The above findings and conclusions may be used to support various exemplary aspects of the invention. In one aspect of the present findings provides a method of promoting muscle growth, decreasing fat deposits or improving feed efficiency of livestock animals, including birds and farmed fish, which comprises administering to said animal an effective amount of the pure or substantially pure RR-enantiomer of the adrenergic beta-receptor agonist ractopamine or a pharmaceutically acceptable salt thereof, while reducing concomitant liability of adverse effects associated with racemic ractopamine.

The animals in this aspect are raised for consumption by humans. wherein the animals are raised for human meat production.

The daily dose of said beta-receptor agonist is from about 0.1 mg to about 500 mg per day.

In some aspects effective amount of the pure or substantially pure RR-enantiomer of ractopamine or a pharmaceutically acceptable salt thereof is administered for promoting muscle growth of warm-blooded animals or farmed fish.

In other aspects, an effective amount of the pure or substantially pure RR-enantiomer of ractopamine or a pharmaceutically acceptable salt thereof is administered for decreasing fat deposits of warm-blooded animals or farmed fish.

In other aspects, an effective amount of the pure or substantially pure RR-enantiomer of ractopamine or a pharmaceutically acceptable thereof is administered to warm-blooded animals or farmed fish for improving feed efficiency.

The adverse effect is varied and can be stress. The adverse effect can be stress and concomitant tachycardia and increased mortality.

The adverse effect can be stress and concomitant decrease in meat quality.

Another aspect is a feedstuff composition comprising the admixture with protein-containing feed materials of the pure or substantially pure RR-enantiomer of ractopamine.

The feedstuff contains from about 1 to 500 grams of the pure or substantially pure RR-enantiomer of ractopamine per ton of feed.

The feedstuff can also contain an effective amount of an antibacterial compound for the prevention and control of bacterial infections.

The feedstuff can also contain an effective amount of an antibacterial compound for further improved gain of muscle weight, for further decreased fat deposits and/or for further improved feed efficiency.

Examples of the animals are cattle, swine, horses, sheep, goats, chicken, turkeys and ostriches. Examples of the farmed fish are barramundi, carp species, cod species, perch, salmon, trout, and tilapia

The animal is an animal suffering from or having a propensity for stress.

In some aspects of the methods an effective amount of the pure or substantially pure RR-enantiomer of ractopamine or a pharmaceutically acceptable salt thereof is administered to a warm-blooded animal in a concentration of from about 1 ppm to 500 ppm in feed.

In other aspects of the method an effective amount of the pure or substantially pure RR-enantiomer of ractopamine or a pharmaceutically acceptable salt thereof is administered to a warm-blooded animal in a concentration of 1 ppm to 500 ppm in drinking water.

In other aspects of the method the effective amount of the pure or substantially pure RR-enantiomer of ractopamine or a pharmaceutically acceptable salt thereof is administered to a warm-blooded animal is as a parenteral dose of from about 0.05 to 100 mg daily of the pure or substantially pure RR enantiomer of ractopamine.

The optical purity of the RR-enantiomer of ractopamine is greater than approximately 55% by weight, or greater than approximately 80% by weight, or greater than approximately 90% by weight, or greater than approximately 95% by weight, or greater than approximately 98% by weight.

Another aspect includes a method of reducing body fat and/or body weight in obese mammals in need thereof, comprising administering to said obese mammal a therapeutically effective amount of the adrenergic beta-receptor agonist ractopamine or a pharmaceutically acceptable salt thereof.

Another aspect includes a method of reducing body fat and/or body weight in obese mammals in need thereof, comprising administering to said obese mammal a therapeutically effective amount of the pure or substantially pure RR-isomer of the adrenergic beta-receptor agonist ractopamine or a pharmaceutically acceptable salt thereof.

Another aspect includes a method of reducing body fat and/or body weight in obese mammals in need thereof, comprising administering to said obese mammal a therapeutically effective amount of the pure or substantially pure RR-isomer of the adrenergic beta-receptor agonist ractopamine or a pharmaceutically acceptable salt thereof, while minimizing or eliminating the side effects residing in the corresponding distomeric RS-, SR- and SS-enantiomers.

Although the invention has been described with reference to certain preferred embodiments, it will be appreciated that many variations and modifications may be made within the scope of the broad principles of the invention. Hence, it is intended that the preferred embodiments and all of such variations and modifications be included within the scope and spirit of the invention, as defined by the following claims.

1. A method of promoting muscle growth, decreasing fat deposits or improving feed efficiency in livestock animals comprising administering to said animal an effective amount of a pure or substantially pure RR enantiomer of ractopamine or a pharmaceutically acceptable salt thereof, wherein said administration produces fewer adverse effects than the adverse effects associated with administration of a similar dose of a racemic ractopamine.

2. The method of claim 1, wherein the meat from said animals is for human consumption.

3. The method of claims 1, wherein the daily dose of said RR-enantiomer of ractopamine is from about 0.01 mg to about 500 mg per day.

4. The method of claim 1, wherein the animal is a warm-blooded animal or farmed fish.

5. The method of claim 1, wherein said administration reduces stress in said animals as compared to administration of a similar dose of a racemic mixture of ractopamine.

6. The method of claim 1, wherein said administration reduces stress and concomitant tachychardia and increased mortality in said animals as compared to administration of a similar dose of a racemic mixture of ractopamine.

7. The method of claim 1, wherein said administration reduces stress and a concomitant decrease in meat quality in said animals as compared to administration of a similar dose of a racemic mixture of ractopamine.

8. The method of claim 1, wherein said animal is a warm-blooded animal selected from the group consisting of cattle, swine, horses, goat, sheep, chicken, turkeys, and ostriches.

9. The method of claim 1, wherein said animal is a farmed fish selected from the group consisting of barramundi, a carp species fish, a cod species fish, perch, salmon, trout and tilapia.

10. The method of claim 1, wherein said effective amount of said pure or substantially pure RR-enantiomer of ractopamine or pharmaceutically acceptable salt thereof is administered to said animal in a concentration of from about 1 ppm to about 500 ppm of a feed composition.

11. The method of claim 1, wherein said effective amount of said pure or substantially pure RR-enantiomer of ractopamine or pharmaceutically acceptable salt thereof is administered to said animal in a concentration of from about 1 ppm to about 500 ppm in drinking water.

12. The method of claim 1, wherein said effective amount of said pure or substantially pure RR-enantiomer of ractopamine or pharmaceutically acceptable salt thereof is administered to said animal as a dose of from about 0.05 to 100 mg per day, wherein said dose is administered parenterally, rectally or topically or as an aerosol formulation.

13. The method of claim 1, wherein the optical purity of the RR-enantiomer of ractopamine is between about 55% by weight and 98% by weight.

14. A method of reducing body fat and/or body weight in an obese mammal comprising administering to said mammal a therapeutically effective amount of ractopamine or a pharmaceutically acceptable salt thereof.

15. The method of claim 14, wherein said ractopamine is pure or substantially pure RR-isomer of ractopamine or a pharmaceutically acceptable salt thereof.

16. The method of claim 15, wherein administration of said pure or substantially pure RR isomer of ractopamine or pharmaceutically acceptable salt thereof reduces body fat and/or body weight while minimizing or eliminating the side effects associated with administration of an RS-, an SR, or an SS-enantiomer of ractopamine.

17. A feedstuff composition comprising protein-containing feed materials and a pure or substantially pure RR-enantiomer of ractopamine.

18. The feedstuff composition of claim 17, wherein said composition comprises from about 1 to about 500 grams of pure or substantially pure RR-enantiomer of ractopamine per ton of feed material.

19. The feedstuff composition of claim 17, further comprising an effective amount of an antibacterial compound for the prevention or reduction of bacterial infections.

20. The feedstuff composition of claim 16, wherein the composition comprising said antibacterial compound produces an improved gain of muscle weight and/or a further decrease in fat deposits, and/or an improved feed efficiency as compared to a feedstuff composition containing ractopamine in the absence of an antibacterial compound.
